(19) United States
(12) Patent Application Publication
(10) Pub. No.: US 2026/0157853 A1
(43) Pub. Date: Jun. 11, 2026

(54) IMPLANT FOR AN AREAL TREATMENT OF A BONE DEFECT

(71) Applicant: Karl Leibinger Asset Management GmbH & Co. KG, Mühlheim an der Donau (DE)

(72) Inventors: Adem Aksu, VS-Schwenningen (DE); Lena Schaarschmidt, Aldingen (DE)

(21) Appl. No.: 19/416,394

(22) Filed: Dec. 11, 2025

(30) Foreign Application Priority Data
Dec. 11, 2024 (DE) 102024137112.9

(51) Int. Cl. A61F 2/28; A61F 2/30; A61L 27/06; A61L 27/16; A61L 27/18; B33Y 80/00

(52) U.S. Cl. CPC A61F 2/2875; A61F 2/28; A61F 2/30749; A61F 2/30771; A61F 2/3094; A61L 27/06; A61L 27/16; A61L 27/18; B33Y 80/00; A61F 2002/30014; A61F 2002/30065; A61F 2002/3012; A61F 2002/3013; A61F 2002/30317; A61F 2002/30604; A61F 2002/3092; A61F 2310/00023; A61L 2430/02

(57) ABSTRACT

An implant (I) for an areal treatment of a bone defect, includes a lattice structure (GS) having a top side (OS) and an underside (US) facing away from the top side (OS) and on which the implant (I) is fixable to bone. The lattice structure (GS) has at least one first support section (TA1) formed of a first biocompatible material. Furthermore, the lattice structure (GS) has at least one second support section (TA2), which has a lower rigidity compared to the at least one first support section (TA1), in that the at least one second support section (TS2) is formed of a second biocompatible material having a lower material rigidity than the first material.

### CROSS-REFERENCE TO RELATED APPLICATION

The present application is related and has right of priority to German Patent Application No. DE 102024137112.9 filed on Dec. 11, 2024, which is incorporated by reference in its entirety for all purposes.

### TECHNICAL FIELD

The invention relates generally to an implant for an areal treatment of a bone defect, in particular a bone defect in the area of the thorax or of the cranium.

### BACKGROUND

In the field of implants, devices are known that are used for the areal treatment of bone defects. Such an implant frequently has a lattice structure, which is at least partially flexible and thus enables adaptation to different shapes. Implants designed in this way can be used in the thorax area and, in particular, in place of rib plates in the case of rib fractures, so that, by being arranged over a large area on the thorax, areas between different ribs are also covered. Due to the lattice structure, internal organs are protected while, at the same time, sufficient flexibility is achieved for enabling sufficient mobility of the thorax.

Furthermore, implants having a lattice structure in the area of the cranium are provided for treating skull defects, wherein the at least partially flexible lattice structure enables the implant to be adapted to curvatures of the skull and additionally ensures sufficient elasticity. An implant for areal treatment frequently has a mesh-like support section, which forms the lattice structure and is fixated to the bone and used for stabilization.

DE 197 46 396 A1 describes an implant provided for an areal treatment of bone defects in the area of the cranium. The implant has a lattice structure, which is composed in a mesh-like manner of a plurality of segments which are closed and connected to one another. The lattice structure can be fastened at an underside in the corresponding area of the cranium for fixing bone portions or for bridging bone defects, wherein the fastening can be carried out by guiding a bone screw through each individual segment. The interconnected segments form a support section in the lattice structure, via which the bone portions and the bone defect can be stabilized. The lattice structure is produced by etching titanium.

### BRIEF SUMMARY

Example aspects of the present invention provide an implant, via which an areal treatment of a bone defect can be carried out, wherein, when this implant is used, the risk of tissue irritations is also to be reduced.

According to example aspects of the invention, an implant includes a lattice structure having a top side and an underside facing away from the top side and at which the implant is to be fixated to bone. The lattice structure has at least one first support section, which is formed of a first biocompatible material.

The implant according to example aspects of the invention is provided for an areal treatment of a bone defect, wherein this is, in particular, a bone defect in the area of the thorax or the cranium. For this areal treatment, the implant is equipped with a lattice structure, which is preferably flat. The lattice structure has a top side and an underside, which face away from one another and of which the underside is the side with which the implant is placed and fixated facing the bone defect.

An “areal” treatment is to be understood within the meaning of the invention, in particular, to mean that a treatment area is covered by the implant when applied on a bone defect. Within the framework of example aspects of the invention, the implant can be specifically provided for use in this areal treatment to connect bone segments of a fractured bone and/or to establish connections between different bones and/or to cover bone defects. A treatment area to be covered by the implant according to example aspects of the invention can be in the range of a few square millimeters to many square centimeters depending on the specific use case, i.e., in particular whether an application is intended in the area of the thorax or the cranium.

Example aspects of the invention provide that the lattice structure has, in addition to the at least one first support section, at least one second support section, which has a lower rigidity compared to the at least one first support section, in that the at least one second support section is made of a second biocompatible material having a lower material rigidity than the first material.

In other words, the lattice structure of the implant according to example aspects of the invention is therefore composed of support sections having rigidities that deviate from one another, in that, in addition to the at least one first support section, at least one second support section is provided in the lattice structure, which has a lower rigidity compared to the at least one first support section. The at least one first support section formed of a first biocompatible material and the at least one second support section formed of a second biocompatible material, wherein the lower rigidity of the at least one second support section compared to the at least one first support section is achieved in that the second material has a lower material rigidity than the first material.

Such an example embodiment of an implant has the advantage that, due to the portional design of the lattice structure made of support sections having different rigidity, the implant can be optimally adapted to the requirements of the treatment. Via the at least one first, more rigid support section, high stability can thus be achieved locally in a targeted manner, whereas a higher flexibility of the lattice structure is achievable locally via the at least one second support section due to the lower rigidity compared to the first support section, whereby the lattice structure can be more easily adapted in terms of its shape to a defined placement region on bone. In the implant according to example aspects of the invention, different regions can be created by constructing the lattice structure from the two types of support sections, namely at least one region of high stability due to the higher rigidity of the respective first support section and at least one region of higher flexibility and resilience due to the lower rigidity of the respective second support section. The rigidities, which deviate from one another, can be reliably achieved by forming the support sections from the materials having different material rigidities. Furthermore, it is possible to provide further material-dependent properties on the support sections depending on the selected material.

The “rigidity” of the respective support section is to be understood, within the meaning of the invention, to mean in particular the resistance of the respective support section to a deformation brought about by external loading. The “material rigidity” of the respective material is preferably understood to mean a material-mechanical property defined by the ratio of the acting tension and the resulting expansion, wherein material characteristic values that are relevant are, in particular, the modulus of elasticity and the shear modulus.

Due to the portional design, the lattice structure is flexible at least in the region of the at least one second support section, whereby the lattice structure is given, in particular, three-dimensional formability in order to be able to easily adapt the shape of the implant to a defined placement region on bone. The lattice structure of the implant according to example aspects of the invention is thus adaptable, due to its flexible design, in particular, to a curved bone defect and to a bone defect having an irregular shape. Preferably, the lattice structure is also moldable in the region of the at least one first support section, however, wherein the at least one first support section is more resistant to deformations due to the higher rigidity compared to the at least one second support section. In general, the lattice structure can be designed, within the framework of example aspects of the invention, to be adapted by the attending surgeon in terms of size and shape by removing parts of the lattice structure and thus parts of the support sections, for example by cutting the lattice structure to size.

The implant according to example aspects of the invention is provided for use in the treatment of the bone defect, in particular, to stabilize bone. For this purpose, the lattice structure is preferably fixated to bone in the region of the bone defect to be treated, wherein this fixation to bone can be present in the form of a fixation to a plurality of bone segments of a bone or to a plurality of regions of a bone or also to different bones. Preferably, the lattice structure is equipped for this purpose with a plurality of fixation points, which are present, in particular, in the form of a respective screw connection point at which the implant is fixable via a respective bone screw. In particular, each of the support sections is provided with a plurality of such fixation points. Depending on the specific treatment case and size of the treatment area, the lattice structure can be composed of one or a plurality of first support sections and one or a plurality of second support sections.

According to one example embodiment of the invention, the at least one second support section in the lattice structure is arranged between at least two first support sections. The at least one second support section couples the at least two first support sections to one another. Advantageously, via the at least one second support section between a plurality of rigid support sections, a more flexible intermediate region can thereby be formed, in which the shape of the implant is provided with better adaptability and lower levels of tissue irritation of adjacent tissue are induced. This allows bone regions to be stabilized in a targeted manner via the first support sections and for these stabilized bone regions to be coupled to one another via the intermediate, second support section. Due to the lower rigidity of the second support section, the risk of tissue irritation is reduced locally in a targeted manner and, nevertheless, a certain mobility of the stabilized bone regions with respect to one another is created. When the implant is used in the thorax area, for example, a targeted local increase in the flexibility of the implant can be achieved in order to simplify the continuous movement of the thorax due to the breathing or other types of movement of the patient.

According to one possible example embodiment of the invention, the lattice structure is modular, in that the at least one first support section and the at least one second support section are fastened to one another. Preferably, this fastening is achieved by an integral bond between the support sections. In a development of this possible example embodiment, the at least one second support section covers the at least one first support section in the region of the respective fastening in a direction extending transversely to the top side and the underside. Advantageously, due to this coverage, a higher load-bearing capacity of the connection between the support sections can be created. Most particularly preferably, the at least one second support section engages around the at least one first support section in the region of the respective fastening on each side with protruding connecting segments, whereby the load-bearing capacity of the connection is further increased.

According to a further example embodiment of the invention, at least one of the support sections has a mesh structure formed by closed segments which are connected to one another via intermediate segments. A construction is therefore achieved in the respective support section, in which mobility and thus flexibility is created in the region of the respective support section. Preferably, the closed segments are ring-shaped, wherein, within the framework of example aspects of the invention, the segments can also have shapes deviating therefrom, for example the shape of a polygon. The segments are connected to one another in the mesh structure via intermediate segments, which can be linear or non-linear. It would also be conceivable that the intermediate segments each define a closed segment by being fastened to one another. In the present example case, either the at least one first support section or the at least one second support section or the at least one first support section as well as the at least one second support section are each provided with a mesh structure of this type.

Alternatively or additionally, at least one of the support sections is plate-shaped. Most particularly preferably, a plate-shaped structure is created in the at least one first support section in order to achieve greater rigidity here. In principle, however, the at least one second support section could also be plate-shaped.

In a development of example aspects of the invention, at least one of the support sections has been produced by an additive manufacturing process. One possibility within the meaning of example aspects of the invention is, in particular, production within the framework of a 3D printing process. Particularly preferably, the at least one first support section has been formed by additive manufacturing, in particular by 3D printing.

More preferably, the second biocompatible material has a lower hardness than the first material. This has the advantage that the at least one second support section is therefore softer than the at least one first support section, whereby the risk of tissue irritation in the region of the respective second support section is reduced.

According to one possible example embodiment of the invention, the first material is a metal or a metal alloy, in particular titanium or a titanium alloy. More particularly preferably, however, the first material is a plastic, wherein this plastic is, in particular, a polymer, preferably a thermoplastic, and, particularly preferably, polyether ether ketone (PEEK). This is the case, in particular, because polyether ether ketone is distinguished by very high biocompatibility and high material rigidity, such that high stability of the lattice structure can therefore also be achieved via the respective first support section.

The second material is preferably a plastic, in particular a polymer, preferably a thermoplastic and, particularly preferably, polyethylene (PE), for example ultra high molecular weight polyethylene (UHMWPE) or high density polyethylene (HDPE). As a result, the lower rigidity of the respective second support section to be provided compared to the respective first support section can be achieved in a reliable way. In particular, the polyethylene forms the respective second support section as a non-porous layer, as a result of which, despite the lower rigidity, sufficient stability can be achieved and, in addition, a smooth surface of the respective second support section can be obtained.

Most particularly preferably, the two aforementioned example variants are jointly implemented, wherein, in order to produce the implant according to the invention, initially the at least one support section made of polyether ether ketone (PEEK) is manufactured. This is carried out, particularly preferably, within the framework of additive manufacturing. Then, for cleaning purposes, a plasma treatment/plasma activation of the at least one first support section is preferably carried out before the at least one support section is placed together with polyethylene powder layer-by-layer in a negative mold. In a subsequent pressing process, the polyethylene (PE) is then heated together with the polyether ether ketone (PEEK), whereby the polyethylene is fused with the polyether ether ketone, forming the at least one second support section. As a result, a loadable bond between polyether ether ketone and polyethylene can be obtained.

According to a further possible example embodiment of the invention, a cover layer is at least partially applied on at least one of the support sections, which cover layer has a lower hardness compared to the respective support section. Due to the at least partial application of such a cover layer on the respective support section and the lower hardness of this cover layer compared to this support section, the risk of skin or tissue irritations can be reduced. This is the case because, due to the at least partial coverage of the respective support section with the respective cover layer, a softer surface of the implant is obtained in the respective area due to the lower hardness of the cover layer.

In particular, the cover layer is applied at least onto the at least one first support section. This is achieved, in particular, when the at least one first support section has been produced within the framework of an additive manufacturing process. As a result, irregularities and edges of this support section can be covered via this cover layer and thus a softer surface of the implant can be created in this area.

Within the meaning of the invention, “hardness” is to be understood as the mechanical resistance that is applied against mechanical penetration. Accordingly, the cover layer therefore applies a lower mechanical resistance to a mechanical penetration than is the case with the respective support section. The cover layer can therefore be described as softer compared to the respective support section.

In the aforementioned possible example embodiment, the respective support section is at least partially coated with the cover layer, i.e. the respective support section can therefore be provided with each respective cover layer on one or more portions thereof or also around the entire periphery thereof.

According to one example embodiment of the invention, the respective support section is coated with the respective cover layer on at least one subsection on the top side of the lattice structure. In this case, the cover layer can therefore be partially applied on the top side on the respective support section, whereby the respective support section is then partially uncoated on the top side. Alternatively, the respective support section is completely coated with the cover layer on the top side of the lattice structure, such that the respective support section is then completely coated with the cover layer on the top side. Advantageously, the respective support section is therefore either covered in certain regions in a targeted manner or completely covered on the top side. In the latter case, tissue irritations are therefore completely prevented on the top side, whereas, in the sectional coating, contact areas with tissue and/or irregularities of the respective support section, for example edges or the like, can be intentionally designed to be softer with the aid of the cover layer.

Alternatively or additionally, the respective support section can be coated with each respective cover layer on at least one subsection on the underside of the lattice structure. The respective support section is therefore partially provided with the cover layer on the underside of the lattice structure in this case, as a result of which the respective support section is therefore partially uncoated on the underside. The respective support section could also be completely coated with the cover layer on the underside of the lattice structure, however, whereby the respective support section is then completely covered with the cover layer on the underside. In both cases, a softer surface is at least partially obtained on the underside of the implant by either partially or completely coating the respective support section on the underside. As a result, tissue irritations on the underside of the implant can be reduced. In the case of the partial coating of the respective support section, this is coated in particular intentionally in certain contact regions with tissue and/or at irregularities, for example edges or the like.

The aforementioned example developments of the invention can be achieved alternatively or additionally, as a result of which the respective support section can be partially or completely coated on the top side, partially or completely on the underside, or both partially or completely on the top side, and partially or completely on the underside.

In combination, an example design of the implant is also conceivable, in which the respective support section is completely encapsulated in the cover layer. In this case, the respective support section is then therefore completely surrounded by the associated cover layer, i.e. completely coated.

Within the framework of example aspects of the invention, the cover layer can be porous. This has the advantage that, due to this porous design, a particularly low hardness of the respective cover layer is achieved. On the other hand, the possibility is thereby created that tissue can grow into the implant and vascularization is possible. In a development of this example embodiment, the respective support section is then at least partially embedded into the porous cover layer. Advantageously, an even, soft surface can thereby be obtained in the corresponding area.

Alternatively, the cover layer can be non-porous. As a result, a very smooth and simultaneously soft surface can be obtained in this area, whereby tissue irritations can be largely avoided. Due to the non-porous design of the cover layer, a growth of tissue thereon can be prevented. In particular, a respective structuring of the cover layer corresponds to a structuring which the respective support section has in the region of the coating with the cover layer. This has the advantage that a structure of the respective support section is thereby retained.

Most particularly preferably, the lower hardness of the applied cover layer is achieved in that the applied cover layer has been formed from a biocompatible material having a lower hardness than the material of the respective support section. In the particularly preferred possible example embodiment of the invention, in which the at least one first support section consists of polyether ether ketone and the at least one second support section consists of polyethylene, the applied cover layer is also made of polyethylene. The cover layer can then be formed only in the region of the at least one first support section or also additionally formed on the second support section, which also consists of polyethylene, in order to create an even softer surface of the implant here, for example, due to a porous structure of the cover layer.

### BRIEF DESCRIPTION OF THE DRAWINGS

Advantageous embodiments of the invention, which are explained in the following, are shown in the drawings. In the drawings:

FIGS. 1 and 2 show schematic views of an implant corresponding to a first example embodiment;

FIG. 3 shows a schematic view of an implant according to a second possible example embodiment;

FIG. 4 shows a schematic view of an implant according to a third example embodiment;

FIG. 5 shows a schematic view of an implant according to a fourth possible example embodiment; and

FIGS. 6 through 8 show schematic views of possible example modifications of the implants from FIGS. 1 through 5.

### DETAILED DESCRIPTION

Reference will now be made to embodiments of the invention, one or more examples of which are shown in the drawings. Each embodiment is provided by way of explanation of the invention, and not as a limitation of the invention. For example, features illustrated or described as part of one embodiment can be combined with another embodiment to yield still another embodiment. It is intended that the present invention include these and other modifications and variations to the embodiments described herein.

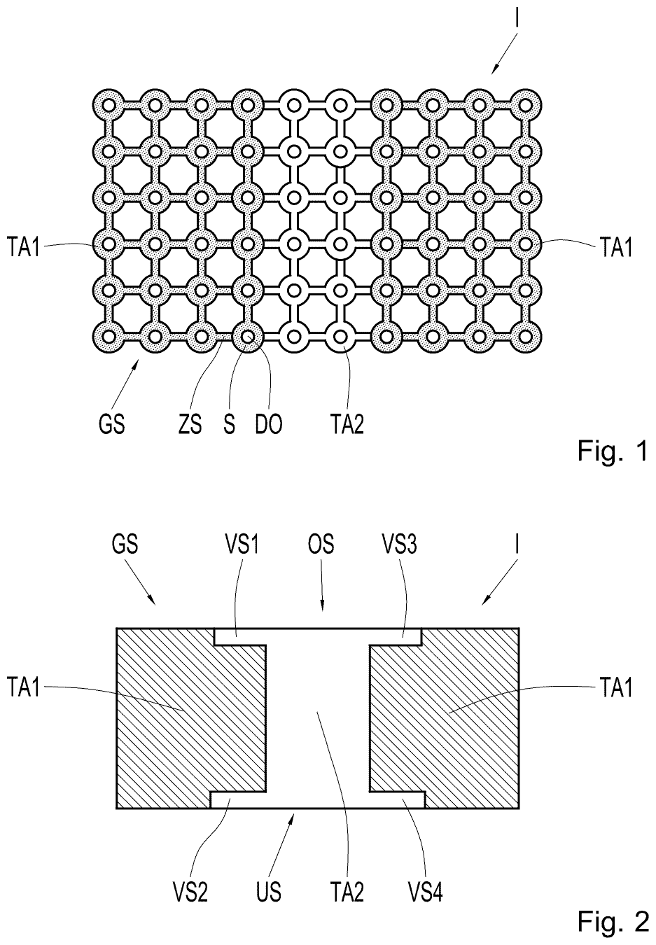

FIGS. 1 and 2 show schematic views of an implant I, which is provided for an areal treatment of bone defects and, in particular, in the thorax area. As is apparent in FIG. 1, the implant I has a lattice structure GS, which is formed by a plurality of support sections TA1 and TA2. In the lattice structure GS, a support section TA2 is arranged between two support sections TA1, which are coupled to one another via the respective intermediate support section TA2. For this purpose, the support section TA2 is connected in the lattice structure GS on each side to the support sections TA1, forming the flat lattice structure GS.

As is apparent with reference to FIG. 1, the support sections TA1 and TA2 each have a mesh structure, in that the respective support section TA1 or TA2 is composed of ring-shaped segments S and intermediate segments ZS, which are designed as linear intermediate pieces and via which the ring-shaped segments S are connected to one another. The ring-shaped segments S in the respective support section TA1 or TA2 each form a through-hole DO in which one bone screw each for fixating the implant I can be received. Using the bone screws, the implant I can be fixated to bone in the region of the bone defect to be treated. Specifically, bone segments or bone portions of one rib bone or a plurality of rib bones and/or a plurality of rib bones can be connected to one another in the thorax area, wherein the implant I then brings about corresponding stabilization via the lattice structure GS.

The support sections TA1 located on each side of the support section TA2 each consist of a first biocompatible material in the form of polyether ether ketone (PEEK), wherein the respective support section TA1 has been produced within the framework of an additive manufacturing process and, in particular, within the framework of 3D printing. However, the respective intermediate support section TA2 connecting the two support sections TA1 to one another in the implant I is formed from a second biocompatible material, which is polyethylene (PE) in the present example case.

As is apparent from FIG. 2, coverage has been implemented in the region of the connection of the intermediate support section TA2 to the respective adjacent support section TA1, which coverage extends in a direction transverse to a top side OS and an underside US of the lattice structure GS. The top side faces away from the underside US at which the implant I is to be placed on and fixated to the bone defect to be treated. To implement the respective coverage, the intermediate support section TA2 is equipped with connecting segments VS1 and VS2, and VS3 and VS4, respectively, at ends at which the connection is established to the respective adjacent support section TA1. These connecting segments VS1 through VS4 protrude from the intermediate support section TA2 in pairs in the manner of a clamp, wherein the support section TA2 then engages around the respective support section TA1 with the associated connecting segments VS1 and VS2, and VS3 and VS4, respectively, in the region of the respective connection.

The connections of the support section TA2 with the support sections TA1 located on each side are each formed by integral joining in the present example case, in that the polyethylene forming the support section TA2 has been fused together with the polyether ether ketone (PEEK) forming the support sections TA1 within the framework of a pressing process under heat. For this purpose, the support sections TA1, after having been additively manufactured, were subjected to a plasma treatment/plasma activation and then placed into a negative mold of the lattice structure GS, wherein the open area located between the support sections TA1 was subsequently filled with polyethylene powder. Within the framework of the pressing process, the polyethylene forms the intermediate support section TA2 and, at the same time, integrally joins this to the support sections TA1 located on each side. A non-porous layer is formed by the polyethylene.

The lattice structure GS of the implant I is flexible, wherein this flexibility is due, on the one hand, to the fact that the intermediate segments ZS in the respective support section TA1 or TA2 enable the ring-shaped segments S to move relative to one another. This flexibility of the lattice structure GS is further increased in the region of the intermediate support section TA2, in that the intermediate support section TA2 has a lower rigidity, due to being made of polyethylene, than the support sections TA1 located on each side thereof. The reason for this is the lower material rigidity of polyethylene compared to the polyether ether ketone of the support sections TA1 located on each side. Due to this design of the lattice structure GS, the implant I can be easily adapted to curvatures of the bones in the region of the bone defect to be treated. On the other hand, when the implant I is in the fixated state, a certain mobility is thereby permitted in order to facilitate movement of the thorax due to the breathing or movement of the patient. Apart from the increased flexibility of the lattice structure GS, the intermediate support section TA2 also ensures that tissue irritations are reduced, due to the lower rigidity and the lower hardness of the polyethylene compared to the polyether ether ketone.

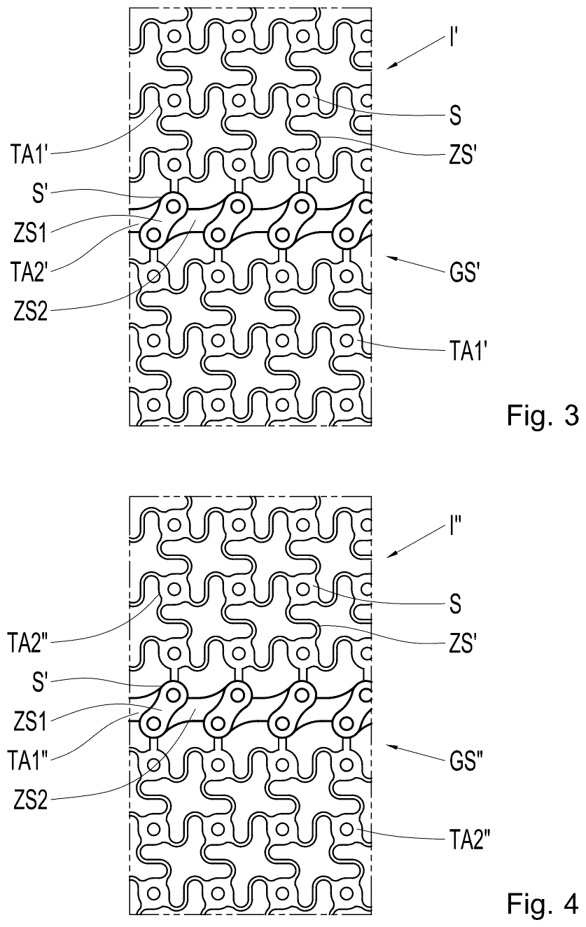

FIG. 3 shows a view of a part of a further implant′, which largely corresponds to the preceding example variant according to FIGS. 1 and 2. This implant I′ also includes a lattice structure GS′, which is formed by a plurality of support sections TA1′ and TA2′. In this lattice structure GS′, support sections TA1′ additively manufactured from polyether ether ketone are connected to one another via a respective intermediate support section TA2′. In contrast to the implant I from FIGS. 1 and 2, the intermediate support section TA2′ is produced in a plate shape, however. In this plate-shaped design, ring-shaped segments S′ are connected to one another in pairs via intermediate segments ZS1 and, as a result, form figure eight-shaped structures, wherein these structures are then connected to one another via further, web-like intermediate segments ZS2 so as to form the support section TA2′. Furthermore, in the support sections TA1′ located on each side of the respective support section TA2′, a mesh structure deviating from the implant I from FIGS. 1 and 2 is selected, in that the segments S are then connected to one another via respective non-linear, S-shaped intermediate segments ZS′. For the rest, the example embodiment according to FIG. 3 corresponds to the example variant according to FIGS. 1 and 2, such that reference is made to the descriptions thereof. In particular, the connection of the intermediate support section TA2′ to the respective adjacent support section TA1′ is also implemented with coverage.

In the implant I″ according to example aspects of the invention shown in FIG. 4, however, in comparison to the example variant according to FIG. 3, a mirrored design is selected such that, in a lattice structure GS″, a respective support section TA1″ additively manufactured from polyether ether ketone now has the plate-shaped design described with reference to FIG. 3, whereas support sections TA2″ made of polyethylene are then each provided with the mesh structure described with reference to FIG. 3. Each of the support sections TA2″ then connects the respective support section TA1″ in the lattice structure GS″ to further, plate-shaped support sections made of polyether ether ketone. For the rest, the example variant according to FIG. 4 corresponds to the preceding example variant according to FIG. 3, such that reference is made to the description thereof.

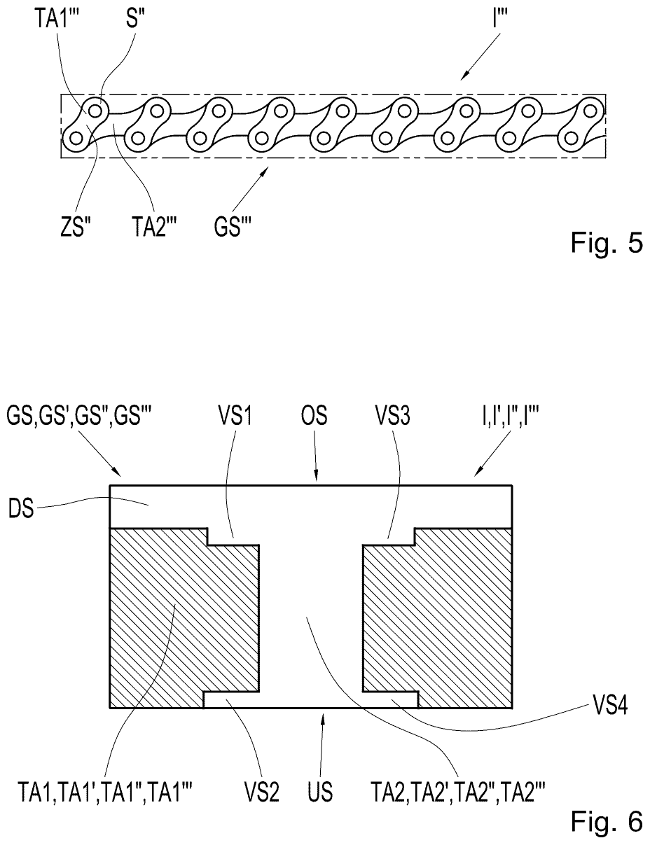

Moreover, FIG. 5 shows an example embodiment according to the invention of an implant I′″, wherein this implant I′″ largely corresponds to the example variant according to FIG. 3. The difference is that, in the implant I′″, support sections TA″ formed from polyether ether ketone (PEEK) in a lattice structure GS′″ are designed as figure eight-shaped structures, in which ring-shaped segments S″ are connected to one another in pairs via a respective intermediate segment ZS″. The support sections TA2′″ are then coupled to one another via intermediate support sections TA2′″, each of which has a lower rigidity compared to the support sections TA1′″, in that these support sections TA2′″ consist of polyethylene (PE). A connection of the individual support section TA2′″ to the support sections TA1′″ located on each side is carried out in a manner similar to the variant according to FIGS. 1 and 2. For the rest, the example embodiment according to FIG. 5 corresponds to the example variant according to FIG. 3, such that reference is made to the description thereof.

Finally, FIGS. 6 through 8 each show possible example modifications of the implants I, I′, I″, and I′″ from FIGS. 1 through 5. In the possible embodiment according to FIG. 6, the respective support sections TA1 and TA2; TA1′ and TA2′; TA1″ and TA2″; TA1′″ and TA2′″ are partially coated, in that a cover layer DS is now provided on the top side OS of the respective implant I, I′, I″, and I′″. This cover layer DS is made of polyethylene, wherein the cover layer DS can be porous or non-porous.

The background for applying the cover layer DS on the top side OS of the respective implant I, I′, I″, and I′″ is that a softer surface is to be obtained as a result, in particular in the region of the respective additively manufactured support sections TA1 TA1′, TA1″, and TA1′″. This is the case because the additive manufacturing of the respective support sections TA1, TA1′, TA1″, and TA1″ has the consequence that a raw surface and, in part, also hard edges are produced on the respective support section TA1, TA1′, TA1″, and TA1′″, which can result in tissue irritations once the respective implant I, I′, 1″, and I′″ has been placed in the body of the patient. Furthermore, the structure of the respective support section TA1, TA1′, TA1″, and TA1″ is noticeable through the tissue or the skin of the patient under some circumstances, which can also result in corresponding irritations. This is reduced by forming the cover layer DS of polyethylene, such that it has a lower hardness compared to the respective support sections TA1, TA1′, TA1″, and TA1′″.

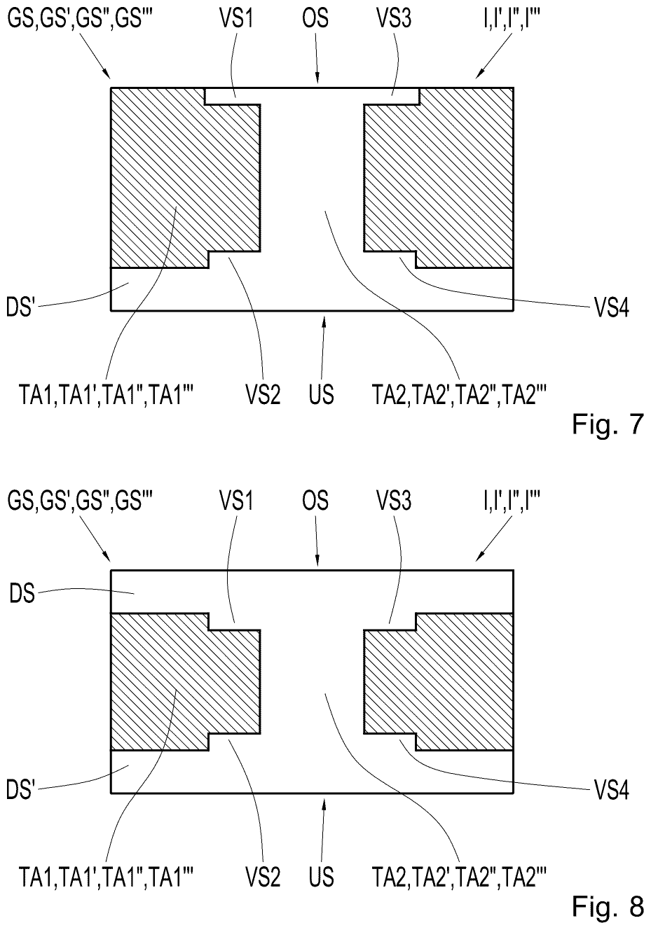

In the possible example modification according to FIG. 7, a cover layer DS' is applied in a manner similar to the possible modification according to FIG. 6, although in this case on the underside US of the respective implant I, I′, I″, and I′″ in order to obtain a softer surface here.

Finally, FIG. 8 shows a further possible example modification of the implants I, I′, I″, and I′″ from FIGS. 1 through 5, wherein the respective implant I, I′, I″, and I′″ is coated both on the top side OS with the cover layer DS and on the underside US with the cover layer DS′. Thus, the respective lattice structure GS, GS′, GS″, or GS′″ of the respective implant I, I′, I″, or I′″ is not sandwiched between the cover layers DS and DS′.

By the embodiments according to example aspects of the invention, an implant can be created in each case, via which a reliable areal treatment of a bone defect is achievable, wherein, when this implant is used, the risk of tissue irritations is also reduced.

Modifications and variations can be made to the embodiments illustrated or described herein without departing from the scope and spirit of the invention as set forth in the appended claims. In the claims, reference characters corresponding to elements recited in the detailed description and the drawings may be recited. Such reference characters are enclosed within parentheses and are provided as an aid for reference to example embodiments described in the detailed description and the drawings. Such reference characters are provided for convenience only and have no effect on the scope of the claims. In particular, such reference characters are not intended to limit the claims to the particular example embodiments described in the detailed description and the drawings.

List of reference characters

| | |
|---|---|
| I, I′, I″, I″′ | implant |
| GS, GS′, GS″, GS″′ | lattice structure |
| TA1, TA2, TA1′, TA2′, TA1″, TA2″, TA1″′, | support sections |
| TA2″′ | |
| S, S′, S″ | segments |
| ZS, ZS1, ZS2, ZS′, ZS″ | intermediate segments |
| DO | through-hole |
| OS | top side |
| US | underside |
| VS1, VS2, VS3, VS4 | connecting segments |
| DS, DS′ | cover layer |

1-14: (canceled)

15. An implant (I; I′; I″; I′″) for an areal treatment of a bone defect, comprising:
a flexible lattice structure (GS; GS′; GS″; GS′″) with a top side (OS) and an underside (US) that faces away from the top side (OS), the lattice structure (GS; GS′; GS″; GS′″) fixable to bone on the underside (US) of the lattice structure (GS; GS′; GS″; GS′″),
wherein the lattice structure (GS; GS′; GS″; GS′″) comprises at least one first support section (TA1; TA1′; TA1″; TA1′″) formed of a first biocompatible material,
wherein the lattice structure (GS; GS′; GS″; GS′″) further comprises at least one second support section (TA2; TA2′; TA2″; TA2′″) formed of a second biocompatible material such that a rigidity of the at least one second support section (TA2; TA2′; TA2″; TA2′″) is less than a rigidity of the at least one first support section (TA1; TA1′; TA1″; TA1′″), the second biocompatible material having a lesser material rigidity than the first biocompatible material.

16. The implant (I; I′; I″; I′″) of claim 15, wherein:
the at least one first support section (TA1; TA1′; TA1″; TA1′″) comprises at least two first support sections (TA1; TA1′; TA1″; TA1′″);
the at least one second support section (TA2; TA2′; TA2″; TA2′″) is arranged in the lattice structure (GS; GS′; GS″; GS′″) between the at least two first support sections (TA1; TA1′; TA1″; TA1′″); and
the at least two first support sections (TA1; TA1′; TA1″; TA1′″) are coupled together.

17. The implant (I; I′; I″; I′″) of claim 15, wherein the lattice structure (GS; GS′; GS″; GS′″) is modular such that the at least one first support section (TA1; TA1′; TA1″; TA1′″) and the at least one second support section (TA2; TA2′; TA2″; TA2′″) are fastened together by an integral bond between the at least one first support section (TA1; TA1′; TA1″; TA1′″) and the at least one second support section (TA2; TA2′; TA2″; TA2′″).

17. The implant (I; I′; I″; I′″) of claim 16, wherein the at least one second support section (TA2; TA2′; TA2″; TA2′″) covers the at least one first support section (TA1; TA1′; TA1″; TA1′″) at a fastening region in a direction extending transversely to the top side (OS) and the underside (US).

18. The implant (I; I′; I″; I′″) of claim 17, wherein the at least one second support section (TA2; TA2′; TA2″; TA2′″) engages around the at least one first support section (TA1; TA1′; TA1″; TA1′″) on each side with protruding connecting segments (VS1, VS2, VS3, VS4) in the fastening region.

19. The implant (I; I′; I″; I′″) of claim 15, wherein at least one of the first and second support sections (TA1, TA2; TA1′, TA2′; TA1″, TA2″; TA1′″, TA2′″) has a respective mesh structure formed by closed segments (S) connected by intermediate segments (ZS, ZS′).

20. The implant (I′; I″; I′″) of claim 15, wherein at least one of the first and second support sections (TA′, TA2′; TA1″, TA2″; TA1′″, TA2′″) is plate-shaped.

21. The implant (I; I′; I″; I′″) of claim 15, wherein at least one of the first and second support sections (TA1, TA2; TA1′, TA2′; TA1″, TA2″; TA1′, TA2′″) is produced by an additive manufacturing process.

22. The implant (I; I′; I″; I′″) of claim 15, wherein a hardness of the second biocompatible material is less than a hardness of the first biocompatible material.

23. The implant (I; I′; I″; I′″) of claim 15, wherein the first biocompatible material is a metal or a metal alloy.

24. The implant (I; I′; I″; I′″) of claim 23, wherein the first biocompatible material is titanium or a titanium alloy.

25. The implant (I; I′; I″; I′″) of claim 15, wherein the first biocompatible material is a plastic.

26. The implant (I; I′; I″; I′″) of claim 25, wherein the first biocompatible material is a thermoplastic.

27. The implant (I; I′; I″; I′″) of claim 25, wherein the first biocompatible material is polyether ether ketone.

28. The implant (I; I′; I″; I′″) of claim 15, wherein the second biocompatible material is a plastic.

29. The implant (I; I′; I″; I′″) of claim 28, wherein the second biocompatible material is a thermoplastic.

30. The implant (I; I′; I″; I′″) of claim 28, wherein the second biocompatible material is polyethylene.

31. The implant (I; I′; I″; I′″) of claim 15, wherein a cover layer (DS; DS′) is at least partially applied on at least one of the first and second support sections (TA1, TA2; TA1′, TA2′; TA1″, TA2″; TA1′″, TA2′″), and a harness of the cover layer is less than a hardness of the at least one of the first and second support sections (TA1, TA2; TA1′, TA2′; TA1″, TA2″; TA1′″, TA2′″).

32. The implant (I; I′; I″; I′″) of claim 31, wherein the at least one of the first and second support sections (TA1, TA2; TA1′, TA2′; TA1″, TA2″; TA1′″, TA2′″) is coated with the cover layer (DS) at least in subsections or completely on the top side (OS) of the lattice structure (GS; GS′; GS″; GS′″).

33. The implant (I; I′; I″; I′″) of claim 31, wherein the at least one of the first and second support section (TA1, TA2; TA1′, TA2′; TA1″, TA2″; TA1′″, TA2′″) is coated with the cover layer (DS′) at least in subsections or completely on the underside (US) of the lattice structure (GS; GS′; GS″; GS′″).
